(19) United States
(12) Patent Application Publication
(10) Pub. No.: US 2005/0038487 A1
(43) Pub. Date: Feb. 17, 2005

(54) Controlling pulse energy of an optical amplifier by controlling pump diode current

(76) Inventor: Richard Stoltz, Plano, TX (US)

Correspondence Address: CHALKER FLORES, LLP, 12700 PARK CENTRAL, STE. 455, DALLAS, TX, 75251, US

(21) Appl. No.: 10/850,325

(22) Filed: May 19, 2004

Related U.S. Application Data
(60) Provisional application No. 60/494,322, filed on Aug. 11, 2003.
(60) Provisional application No. 60/503,578, filed on Sep. 17, 2003.

(51) Int. Cl. A61N 1/00

(52) U.S. Cl. 607/88

(57) ABSTRACT

The present invention includes methods for using optically-pumped amplifiers to control the temperature of the amplifier by controlling pump diode current to avoid operation in the region where performance is seriously degraded by high amplifier temperature. The pulse energy of semiconductor optical amplifiers may also be adjusted by changing the repetition rate of pulse in the amplifier.

### CROSS-REFERENCE TO RELATED APPLICATIONS

This application claims priority to U.S. Provisional Patent Applications, Ser. No. 60/494,322; entitled “Controlling Temperature Of A Fiber Amplifier By Controlling Pump Diode Current,” filed Aug. 11, 2003 (Docket No. ABI-14); and Ser. No. 60/503,578, entitled “Controlling Optically-Pumped Optical Pulse Amplifiers”, filed Sep. 17, 2003 (Docket No. ABI-23).

### TECHNICAL FIELD OF THE INVENTION

The present invention relates in general to the field of light amplification and, more particularly, to the control of temperature in optical amplifiers.

### BACKGROUND OF THE INVENTION

Ablative material removal is especially useful for medical purposes, either in-vivo or on the outside surface (e.g., skin or tooth), as it is essentially non-thermal and generally painless. Ablative removal of material is generally done with a short optical pulse that is stretched amplified and then compressed. A number of types of laser amplifiers have been used for the amplification.

Laser ablation is very efficiently done with a beam of very short pulses (generally a pulse-duration of three picoseconds or less). While some laser machining melts portions of the work-piece, this type of material removal is ablative, disassociating the surface atoms. Techniques for generating these ultra-short pulses are described, e.g., in a book entitled “Femtosecond Laser Pulses” (C. Rulliere —editor), published 1998, Springer-Verlag Berlin Heidelberg New York. Generally large systems, such as Ti:Sapphire, are used for generating ultra-short pulses (USP).

USP phenomenon was first observed in the 1970's, when it was discovered that mode-locking a broad-spectrum laser could produce ultra-short pulses. The minimum pulse duration attainable is limited by the bandwidth of the gain medium, which is inversely proportional to this minimal or Fourier-transform-limited pulse duration. Mode-locked pulses are typically very short and will spread (i.e., undergo temporal dispersion) as they traverse any medium. Subsequent pulse-compression techniques are often used to obtain USP's. Pulse dispersion can occur within the laser cavity so that compression techniques are sometimes added intra-cavity. When high-power pulses are desired, they are intentionally lengthened before amplification to avoid internal component optical damage. This is referred to as “Chirped Pulse Amplification” (CPA). The pulse is subsequently compressed to obtain a high peak power (pulse-energy amplification and pulse-duration compression).

### SUMMARY OF THE INVENTION

It has been found that ablative material removal with a very short optical pulse is especially useful for medical purposes and can be done either in-vivo or on the body surface. An optically-pumped amplifier is a practical device for ablation, e.g., for surgical purposes. It has now been found that an improved way of operating optically-pumped optical amplifiers is achieved in optically-pumped amplifiers by controlling the temperature of the amplifier by varying the pump diode current to avoid operation in the region where performance is seriously degraded by high amplifier temperature. The pulse energy of semiconductor optical amplifiers can be adjusted by changing the repetition rate of pulse in the amplifier.

Furthermore, using the present invention the ablation rate may be controllable independent of pulse energy, the use of more than one amplifier in a parallel train mode (pulses from one amplifier being delayed to arrive one or more nanoseconds after those from another amplifier) allows step-wise control of ablation rate. Thus, the optically-pumped amplifier operating temperature, pulse energy, and ablation rate can all be optimized, independent of one another. In some embodiments this is a man-portable system, e.g., a wheeled cart or a backpack.

Further, the use of more than one amplifier in parallel a train mode (pulses from one amplifier being delayed to arrive one or more nanoseconds after those from another amplifier) allows step-wise control of ablation rate. Thus, the optically-pumped amplifier operating temperature, pulse energy, and ablation rate can all be optimized, independent of one another.

In one embodiment, a pulse of between about 10 picoseconds and one nanosecond wavelength-swept-with-time is generated from a semiconductor oscillator-driven pulse generator, with the initial pulse amplified by a optically-pumped optical amplifier, e.g., a erbium-doped fiber amplifier (or EDFA) or a Cr:YAG amplifier and compressed by an air-path between gratings compressor (e.g., a Tracey grating compressor is an air-grating compressor), with the compression creating a sub-picosecond ablation pulse.

Ablative material removal with a very short optical pulse is especially useful for medical purposes and may be used on either in-vivo or on the body surface. As some materials ablate much faster than others and material is most efficiently removed at pulse energy densities about three times the materials ablation threshold, control of the ablation rate is desirable.

Typically in surgery, the ablation event has a threshold of a fraction of a Joule per square centimeter, but occasionally surgical removal of foreign material may require dealing with an ablation threshold of up to about two Joules per square centimeter. It has been found that control of pulse energy is much more convenient than changing the ablation spot size, and thus control of pulse energy density is desirable. It has further been found that in optically-pumped amplifiers, this can be done by controlling repetition rate. In one embodiment, the ablation rate be also controllable independent of pulse energy. The use of more than one amplifier in parallel a train mode (pulses from one amplifier being delayed to arrive one or more nanoseconds after those from another amplifier) allows step-wise control of ablation rate independent of pulse energy density. At lower desired ablation rates, one or more amplifiers can be shut down.

One embodiment uses of parallel amplifiers to provide faster ablation, whereby providing greater cooling surface area to minimize thermal problems. In addition, one or more of the parallel amplifiers can be shut down, allowing more efficient ablation of a variety of materials with different ablation thresholds, as surfaces are most efficiently ablated at an energy density about three time threshold.

One embodiment of the present invention also includes a method of controlling a optically-pumped amplifier in material removal from a body by optical-ablation, that includes the steps of using an optical oscillator (e.g., a fiber amplifier) in the generation of a series of wavelength-swept-with-time pulses; passing electrical current through at least one pump diode to generate pumping light; optically pumping a optically-pumped amplifier with the pumping light; amplifying the oscillator wavelength-swept-with-time pulse (preferably at least 0.5 nanoseconds in duration to avoid problems from localized hot spots) with the optically-pumped-amplifier; measuring optically-pumped amplifier temperature and controlling the amplifier temperature by controlling the current in the at least one pump diode; controlling pump diode current to control optically-pumped-amplifier temperature; and time-compressing the amplified pulse and illuminating a portion of the body with the time-compressed optical pulse, thereby controlling the pump diode current, which then serves to control the temperature of the optically-pumped amplifier. Preferably, the repetition rate in the optically-pumped-amplifier is controlled to control pulse energy.

In one embodiment, the oscillator, amplifier and compressor are within a man-portable system and the compression is accomplished in an air-path between gratings compressor, the compressed optical pulse has a sub-picosecond duration and the oscillator pulse duration is between 10 picoseconds and one nanosecond. The ablation may be from an outside surface of the body or done inside of the body. Preferably, the pulse energy applied to the body is between 2.5 and 3.6 times ablation threshold of the body portion being ablated.

In another embodiment, the oscillator gives of a series of wavelength-swept-with-time pulses at a fixed repetition rate. In some embodiments, selecting pulses from the oscillator generated series of wavelength-swept-with-time pulses controlling the fraction of pulses selected may be used to achieve finer control of pulse energy.

In some embodiments, the oscillator, amplifier and compressor are within a man-portable system, and/or the compression is done in an air-path between gratings compressor. Preferably, the compressed optical pulse has a sub-picosecond duration, and the amplified pulse has a duration between about ten (10) picoseconds and about one nanosecond. The ablation may be from an outside surface of the body or done inside of the body. In some embodiments, more than one amplifier is used in a mode where amplified pulses from one amplifier are delayed to arrive about one to ten nanoseconds after those from any other amplifier, to allow control of ablation rate independent of pulse energy (after the plume of material being removed by the ablation has substantially dissipated). In other embodiments, more than one amplifier is used in a mode where amplified pulses from one amplifier are delayed to arrive one to ten picoseconds after those from any other amplifier (before the plume of material being removed by the ablation has substantially formed). Preferably, the pulse energy applied to the body is between 2.5 and 3.6 times ablation threshold of the body portion being ablated.

The present invention also include a method of controlling an optically-pumped amplifier in an optical-ablation system, that includes the steps of: generating of a series of wavelength-swept-with-time pulses; passing electrical current through at least one pump diode to generate pumping light; optically pumping a optically-pumped amplifier with the pumping light; amplifying the wavelength-swept-with-time pulses with the optically-pumped-amplifier; measuring optically-pumped amplifier temperature and controlling the amplifier temperature by controlling the current in at least one pump diode; controlling pump diode current to control optically-pumped-amplifier temperature; and time-compressing the amplified pulse.

The amplifying and compressing can be done with a fiber-amplifier and air-path between gratings compressor combination, e.g., with the amplified pulses between 10 picoseconds and one nanosecond, or the amplifying and compressing can be done with a chirped fiber compressor combination, e.g., with the initial pulses between 1 and 20 nanoseconds. In some embodiments a man-portable system includes a wheeled cart or a backpack. The optically-pumped amplifier can be an erbium-doped fiber amplifier, and the air-path between gratings compressor preferably is a Tracey grating compressor.

Preferably, more than one optically-pumped amplifier is used in parallel, or more than one semiconductor optical amplifier is used in parallel. More than one optically-pumped amplifiers may be used with one compressor. High ablative pulse repetition rates are preferred and the total pulses per second (the total system repetition rate) from the one or more parallel optical amplifiers is preferably greater than 0.6 million.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Ablative material removal with a very short optical pulse is especially useful for medical purposes and can be done either in-vivo or on the body surface. It has been found that in optically-pumped amplifiers, control of temperature of an optically-pumped amplifier can be by controlling pump diode current. This control avoids operation in a region where performance is seriously degraded by high optically-pumped-amplifier temperature. The temperature can be measured by a variety of techniques, including thermocouples and thermopiles. The heat sink temperature of a heat-sink-containing optically-pumped-amplifier can also be measured to give an indication of optically-pumped-amplifier temperature.

The pulse energy of semiconductor optical amplifiers can be adjusted by changing the repetition rate of pulse in the amplifier, as it is preferred that ablation rate be controllable independent of pulse energy. The use of more than one amplifier in a parallel train mode, e.g., pulses from one amplifier being delayed to arrive one or more nanoseconds (or 1 to 10 picoseconds) after those from another amplifier, allows step-wise control of ablation rate independent of pulse energy.

It has been found that the combination of optically-pumped-amplifier/a small pulse-compressor enables practical, and significant size reduction, which in turn enables the system to be man-portable, e.g., as a wheeled cart or even in a backpack. A used herein, the term “man-portable” means capable of being moved reasonably easily by one person, e.g., as wheeling a wheeled cart from room to room or possibly even being carried in a backpack. In one embodiment sub-picosecond pulses of between 10 picoseconds and one nanosecond is used, followed by pulse selection, with the selected pulses amplified by a optically-pumped-amplifier (e.g., a erbium-doped fiber amplifier or EDFA) and compressed by an air-path between gratings compressor (e.g., a Tracey grating compressor), with the compression creating a sub-picosecond ablation pulse. Generally, a semiconductor oscillator is used to generate pulses and in some embodiments a semiconductor optical amplifier (SOA) diode preamplifier to amplify the selected pulses before introduction into the optically-pumped amplifier.

While the compressors can be run with inputs from more than one amplifier, reflections from other of the parallel amplifiers can cause a loss of efficiency, and thus should be minimized. The loss is especially important if more than one amplifier is amplifying signals at the same time, as is the case with the SOAs. Thus, each of the parallel SOAs preferably has its own compressor and while the amplified pulses may be put into a single fiber after the compressors, reflections from the joining (e.g., in a star connector) are greatly reduced before getting back to the amplifier. With the fiber amplifiers, however, a nanosecond spacing of sub-nanosecond pulses minimizes amplifying of multiple signals at the same time, and a single compressor is preferably used.

Fiber amplifiers have a storage lifetime of about 100 to 300 microseconds and for ablations purposes, fiber amplifiers have generally been operated with a time between pulses of equal to or greater than the storage lifetime, and thus are generally run a repetition rate of less than 3-10 kHz. Fiber amplifiers are available with average power of 30 W or more.

A moderate-power 5 W average power optically-pumped amplifier has been operated to give pulses of 500 microJoules or more, as energy densities above the ablation threshold are needed for non-thermal ablation, and increasing the energy in such a system, increases the ablation rate in either depth or allows larger areas of ablation or both. We, however, run the optically-pumped amplifier with a time between pulses of a fraction (e.g., one-half or less) of the storage lifetime and use a smaller ablation spot. Preferably our spot is less than about 50 microns in diameter. Preferably, a scan of a smaller spot is performed to get a larger effective ablation area.

Another embodiment uses a parallel optically-pumped amplifiers to generate a train of pulses to increase the ablation rate by further increasing the effective repetition rate (while avoiding thermal problems and allowing control of ablation rate by the use of a lesser number of operating optically-pumped amplifiers). Alternatively, a SOA preamplifier to amplify the initial pulse before splitting to drive multiple parallel optically-pumped amplifiers and another SOA before the introduction of the signal into each optically-pumped amplifier (which allows rapid shutting down of individual optically-pumped amplifiers). Further, the pulses are generally operated with pulses at about three times the ablation threshold for greater ablation efficiency.

The use of a 1 ns pulse with an optically-pumped amplifier and air optical-compressor (e.g., a Tracey grating compressor) typically gives compression with ˜40% losses. At less than 1 ns, the losses in a Tracey grating compressor are generally lower. If the other-than-compression losses are 10%, 2 nanoJoules are needed from the amplifier to get 1 nanoJoule on the target. Preferably, for safety purposes, and for better compressor efficiency, longer wavelength, 1550 nm light is preferably used. The use of greater than 1 ns pulses in an air optical-compressor presents two problems; the difference in path length for the extremes of long and short wavelengths needs to be more 3 cm and thus the compressor is large and expensive, and the losses increase with a greater degree of compression.

Preferably, a semiconductor generated initial pulse is used, e.g., a SOA preamplifier to amplify the initial pulse before splitting to drive multiple amplifiers. A smaller spot is scanned and may be ablated to get a larger effective ablation area, and in many cases the scanned spot is smaller than the above optically-pumped-amplifier case. Alternatively, parallel amplifiers may be used to generate a train of pulses to increase the ablation rate by further increasing the effective repetition rate (while avoiding thermal problems and allowing control of ablation rate by the use of a lesser number of operating amplifiers).

Ablative material removal is especially useful for medical purposes either in-vivo or on the body surface and typically has an ablation threshold of less than 1 Joule per square centimeter, but may occasionally require surgical removal of foreign material with an ablation threshold of up to about 2 Joules per square centimeter. The use of more than one amplifier in parallel train mode (pulses from one amplifier being delayed to arrive a few picoseconds or a few nanoseconds after those from another amplifier. At lower desired powers, one or more amplifiers can be shut off (e.g., by stopping the optical pumping to one or more optically-pumped amplifier), and there will be fewer pulses per train. Thus, with 20 amplifiers there would be a maximum of 20 pulses in a train, but most uses might use only three or four amplifiers and three or four pulses per train.

Generally, the optically-pumped amplifiers may be optically-pumped CW (continuous wave) (and are amplifying perhaps 100,000 times per second in 1 nanosecond pulses). Alternately, non-CW-pumping might be used in operating amplifiers, with amplifiers run in a staggered fashion, e.g., one on for a first half-second period and then turned off for a second half-second period, and another amplifier, dormant during the first-period, turned on during the second period, and so forth, to spread the heat load.

In such systems, control input optical signal power, optical pumping power of optically-pumped amplifiers, timing of input pulses, length of input pulses, and timing between start of optical pumping and start of optical signals to control pulse power, and average degree of energy storage in optically-pumped may be used. The temperature of the optically-pumped amplifiers can be adjusted “independently” of the repetition rate by changing the current through the amplifier diodes (the control system needs to be able to handle some interaction between the two, and as the temperature changes relatively slowly, the repetition rate control preferably reacts relatively rapidly). When multiple pump diodes are used for an optically-pumped amplifier, the control of pump current can be by turning off the current to one or more pump diodes.

Many fiber amplifiers have a maximum power of 4 MW, and thus a 10-microJoule ablation pulse could be as short as 2 picoseconds. Thus, e.g., a 10 picoseconds, 10 microJoule pulse, at 500 kHz (or 50 microJoule with 100 kHz), and, if heating becomes a problem, operating in a train mode and switching fiber amplifiers. Thus, one might rotate the running of ten fiber amplifiers such that only five were operating at any one time (e.g., each on for 1/10 th of a second and off for 1/10 th of a second). Again one can have ten fiber amplifiers with time spaced inputs, e.g., by 1 ns (or 2 picoseconds), to give a train of one to 10 pulses. With 5 W amplifiers operating at 100 kHz (and e.g., 50 microJoules) this could step between 100 kHz and 1 MHz. With 50% post-amplifier optical efficiency and 50 microJoules, to get 6 J/sq. cm on the target, the spot size would be about 20 microns.

Another alternate is to have 20 optically-pumped amplifiers with time spaced inputs, e.g., by 1 nanoseconds, to give a train of one to 20 pulses. With 5 W amplifiers operating at 50 kHz (and e.g., 100 microJoules) this could step between 50 kHz and 1 MHz. With 50% post-amplifier optical efficiency and 100 microJoules, to get 6 Joules/square centimeter on the target, the spot size would be about 33 microns. The amplified pulse might be 50 to 100 picoseconds long. A similar system with 10 optically-pumped amplifiers could step between 50 kHz and 500 kHz.

With 5 W amplifiers operating at 20 kHz (and e.g., 250 microJoules) this could step between 20 kHz and 200 kHz. With 50% post-amplifier optical efficiency and 250 microJoules, to get 6 Joules/square centimeter on the target, the spot size would be about 50 microns. The amplified pulse might be 100 to 250 picoseconds long. A similar system with 30 optically-pumped amplifiers could step between 20 kHz and 600 kHz.

Generally it is the pulse generator that controls the input repetition rate of the optically-pumped amplifiers to tune energy per pulse to about three times threshold per pulse. Another alternative is generating a sub-picosecond pulse and time-stretching that pulse within semiconductor pulse generator to give the initial wavelength-swept-with-time initial pulse. Yet another alternate is to measure light leakage from the delivery fiber to get a feedback proportional to pulse power and/or energy for control purposes. Measurement of spot size, e.g., with a video camera, is useful, and can be done with a stationary spot, but is preferably done with a linear scan.

Thus, it has been found that an optically-pumped-amplifier/compressor can enable practical and significant ablation system size reduction. It was also found that in optically-pumped amplifiers, control of temperature of an optically-pumped amplifier may be achieved by controlling pump diode current. The temperature control avoids operation in a region where performance is seriously degraded by high amplifier temperature. The pulse energy of semiconductor optical amplifiers can be adjusted by changing the repetition rate of pulse in the amplifier, as it is preferred that ablation rate may be controllable independent of pulse energy. The use of more than one amplifier in parallel a train mode (pulses from one amplifier being delayed to arrive one or more nanoseconds after those from another amplifier) allows step-wise control of ablation rate independent of pulse energy. Thus, the optically-pumped-amplifier operating temperature, pulse energy, and ablation rate can all be optimized, independent of one another. In some embodiments this is a man-portable system using a wheeled cart or a backpack.

The camera is preferably of the “in-vivo” type (see “Camera Containing Medical Tool” provisional application No. 60/472,071; Docket No. ABI-4; filed May 20, 2003; which is incorporated by reference herein) using an optical fiber in a probe to convey an image back to, e.g., a vidicon-containing remote camera body. This is especially convenient with a handheld beam-emitting probe.

Smaller ablation areas may be scanned by moving the beam without moving the probe. Large areas may be scanned by moving the beam over a first area, and then stepping the probe to second portion of the large area and then scanning the beam over the second area, and so on. The scanning may be using beam deflecting mirrors mounted on piezoelectric actuators (see “Scanned Small Spot Ablation With A High-Rep-Rate” U.S. Provisional Patent Applications, Ser. No. 60/471.972. Docket No. ABI-6; filed May 20, 2003; which is incorporated by reference herein). Preferably, the system actuators scan over a larger region but with the ablation beam only enabled to ablate portions with defined color and/or area. A combination of time and, area and/or color, can be preset, e.g., to allow evaluation after a prescribed time.

Information of such a system and other information on ablation systems are given in co-pending provisional applications listed in the following paragraphs (which are also at least partially co-owned by, or exclusively licensed to, the owners hereof) and are hereby fully incorporated by reference herein (provisional applications listed by docket number, title and provisional number):

Docket number ABI-1 “Laser Machining” U.S. Provisional Patent Applications, Ser. No. 60/471,922; ABI-4 “Camera Containing Medical Tool” U.S. Provisional Patent Applications, Ser. No. 60/472,071; ABI-6 “Scanned Small Spot Ablation With A High-Rep-Rate” U.S. Provisional Patent Applications, Ser. No. 60/471,972; and ABI-7 “Stretched Optical Pulse Amplification and Compression”, U.S. Provisional Patent Applications, Ser. No. 60/471,971, were filed May 20, 2003;

ABI-8 “Controlling Repetition Rate Of Fiber Amplifier” -U.S. Provisional Patent Applications, Ser. No. 60/494,102; ABI-9 “Controlling Pulse Energy Of A Fiber Amplifier By Controlling Pump Diode Current” U.S. Provisional Patent Applications, Ser. No. 60/494,275; ABI-10 “Pulse Energy Adjustment For Changes In Ablation Spot Size” U.S. Provisional Patent Applications, Ser. No. 60/494,274; ABI-11 “Ablative Material Removal With A Preset Removal Rate or Volume or Depth” U.S. Provisional Patent Applications, Ser. No. 60/494,273; ABI-12 “Fiber Amplifier With A Time Between Pulses Of A Fraction Of The Storage Lifetime”; ABI-13 “Man-Portable Optical Ablation System” U.S. Provisional Patent Applications, Ser. No. 60/494,321; ABI-15 “Altering The Emission Of An Ablation Beam for Safety or Control” U.S Provisional Patent Applications, Ser. No. 60/494,267; ABI-16 “Enabling Or Blocking The Emission Of An Ablation Beam Based On Color Of Target Area” U.S. Provisional Patent Applications, Ser. No. 60/49,4172; ABI-17 “Remotely-Controlled Ablation of Surfaces” U.S. Provisional Patent Applications, Ser. No. 60/494,276 and ABI-18 “Ablation Of A Custom Shaped Area” U.S. Provisional Patent Applications, Ser. No. 60/494,180; were filed Aug. 11, 2003. ABI-19 “High-Power-Optical-Amplifier Using A Number Of Spaced, Thin Slabs” U.S. Provisional Patent Applications, Ser. No. 60/497,404 was filed Aug. 22, 2003;

Co-owned ABI-20 “Spiral-Laser On-A-Disc”, U.S. Provisional Patent Applications, Ser. No. 60/502,879; and partially co-owned ABI-21 “Laser Beam Propagation in Air”, U.S. Provisional Patent Applications, Ser. No. 60/502,886 were filed on Sep. 12, 2003. ABI-22 “Active Optical Compressor” U.S. Provisional Patent Applications, Ser. No. 60/503,659, was filed Sep. 17, 2003;

ABI-24 “High Power SuperMode Laser Amplifier” U.S. Provisional Patent Applications, Ser. No. 60/505,968 was filed Sep. 25, 2003, ABI-25 “Semiconductor Manufacturing Using Optical Ablation” U.S. Provisional Patent Applications, Ser. No. 60/508,136 was filed Oct. 2, 2003, ABI-26 “Composite Cutting With Optical Ablation Technique” U.S. Provisional Patent Applications, Ser. No. 60/510,855 was filed Oct. 14, 2003 and ABI-27 “Material Composition Analysis Using Optical Ablation”, U.S. Provisional Patent Applications, Ser. No. 60/512807 was filed Oct. 20, 2003;

ABI-28 “Quasi-Continuous Current in Optical Pulse Amplifier Systems” U.S. Provisional Patent Applications, Ser. No. 60/529,425 and ABI-29 “Optical Pulse Stretching and Compressing” U.S. Provisional Patent Applications, Ser. No. 60/529,443, were both filed Dec. 12, 2003;

ABI-30 “Start-up Timing for Optical Ablation System” U.S. Provisional Patent Applications, Ser. No. 60/539,026; ABI-31 “High-Frequency Ring Oscillator”, U.S. Provisional Patent Applications, Ser. No. 60/539,024; and ABI-32 “Amplifying of High Energy Laser Pulses”, U.S. Provisional Patent Applications, Ser. No. 60/539,025; were filed Jan. 23, 2004; and

ABI-33 “Semiconductor-Type Processing for Solid-State Lasers”, U.S. Provisional Patent Applications, Ser. No. 60/543,086, was filed Feb. 9, 2004; and ABI-34 “Pulse Streaming of Optically-Pumped Amplifiers”, U.S. Provisional Patent Applications, Ser. No. 60/546,065, was filed Feb. 18, 2004. ABI-35 “Pumping of Optically-Pumped Amplifiers”, was filed 2/26/04.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification, but only by the claims.

1. A method of controlling a fiber amplifier in surgical material removal from a body by optical-ablation, comprising the steps of:
generating of a series of wavelength-swept-with-time pulses;
passing electrical current through at least one pump diode to generate pumping light;
optically pumping a fiber amplifier with the pumping light;
amplifying the wavelength-swept-with-time pulse with the fiber-amplifier;
measuring fiber amplifier temperature and controlling the amplifier temperature by controlling the current in the at least one pump diode;
controlling pump diode current to control fiber-amplifier temperature; and
time-compressing the amplified pulse and illuminating a portion of the body with the time-compressed optical pulse, whereby controlling the pump diode current controls the operating temperature of the fiber amplifier for improved performance.

2. The method of claim 1, wherein the generator, amplifier and compressor are within a man-portable system and the compression is done in an air-path between gratings compressor.

3. The method of claim 1, wherein repetition rate in the fiber-amplifier is controlled to control pulse energy.

4. The method of claim 1, wherein the compressed optical pulse has a sub-picosecond duration.

5. The method of claim 1, wherein the pulse duration during amplification is between 10 picoseconds and one nanosecond.

6. The method of claim 1, wherein the ablation is from an outside surface of the body.

7. The method of claim 1, wherein the ablation is done inside of the body.

8. The method of claim 1, wherein the pulse energy applied to the body is between 2.5 and 3.6 times ablation threshold of the body portion being ablated.

9. A method of controlling an optically-pumped amplifier in an optical-ablation system, comprising the steps of:
generating of a series of wavelength-swept-with-time pulses;
passing electrical current through at least one pump diode to generate pumping light;
optically pumping a optically-pumped amplifier with the pumping light;
amplifying the wavelength-swept-with-time pulses with the optically-pumped-amplifier;
measuring optically-pumped amplifier temperature and controlling the amplifier temperature by controlling the current in at least one pump diode;
controlling pump diode current to control optically-pumped-amplifier temperature; and
time-compressing the amplified pulse.

10. The method of claim 9, wherein the generator, amplifier and compressor are within a man-portable system and the compression is done in an air-path between gratings compressor.

11. The method of claim 9, wherein repetition rate in the optically-pumped amplifier is controlled to control pulse energy.

12. The method of claim 9, wherein the compressed optical pulse has a sub-picosecond duration.

13. The method of claim 9, wherein the pulse duration during amplification is between 10 picoseconds and one nanosecond.

14. The method of claim 9, wherein the ablation is from an outside surface of the body.

15. The method of claim 9, wherein the ablation is done inside of the body.

16. The method of claim 9, wherein the pulse energy applied to the body is between 2.5 and 3.6 times ablation threshold of the body portion being ablated.
